SEQUENCE LISTING (24 sequences)

SEQ ID NO: 1 - DNA, 1807 bp, Homo sapiens
atggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattct

SEQ ID NO: 2 - protein, 564 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 3 - DNA, 1311 bp, Homo sapiens
atggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagacacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaaaataaaataaaataacaagaagaagaaagaaagaaatcccacagaaacagataacctaacacagcccgtgcaacgtattttatacaatgctctgaaaatcatagtctcaatctagacagtcttttcctctagttccctgtattcaaatcccagtgtctaacattcaataaatagctatatgaaatcaaaaaaaaaaaaaaaaaaaaaaaaaaaaaaaaa

SEQ ID NO: 4 - protein, 368 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEENKIK

SEQ ID NO: 5 - DNA, 2000 bp, Homo sapiens
atggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgtagcctccacaaaattgcatcaggccccacaacagctgcttgcatggaatagcagagaataccgcctgctccctccggacagcacactcctgaaaacggggagagaggagccaaacatgctcggtttacactttccttatttactgaatgagtggagggcagagacaggcctggagttacgcacactgagtgccccaacatggaaagaaacatcaggagggacaggaaacgttccctccttaaccaacagttttcaagaccttactggaggcactttattggctacataatcactccatgcggtgggcatcaggcagaatcctggtgcagacccaactttgaggtggaggatttcacagtttctttattttgaacttcccccaggctcccactaattcctctccattctatcctcctccctttcccacaaaagaaaacagaaaggagcagcagtgtttgataccgtatcatccagaggcctggttctctcccattatagggcaaacaagccctggcaagatatttcactcccgccccatgccatgcattaaaaatccaaaattgcctatattccacctgccaagcaagagatgctttcattattgaagttccaaatgtatacctttgagaacagtgccttctcgtcttaaaagagaggtcctcattttgtgagttgggagcagagggaattaaagaaagccatgatgcagggatttggccattcaagccgggcagccttcagagaatgtcatccctaatgacacatgcccgaatgaaggagcggggctgagcttgtcctgccttcgtattgaatgttgcctgtctgcctccttaatagcgggcctctgtgtgagcatttgacaagacttaaaactattcattgaagaaaatggatgatcccccaacaggaagatgcaaccccatgggctgcctgcttgaccacagaagtgcttccagctccagttgctcatctgagaactccccccaccacttgctgttaaaattgttaaaattaaaggccatgttgattgaaaaaaaaaaaaaaaaaaa

SEQ ID NO: 6 - protein, 333 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSLHKIASGPTTAACME

SEQ ID NO: 7 - DNA, 195 bp, Homo sapiens
atgcaagagtcaagatgccggagcaggaatatgaattccctgagccgcgaaatctcaacaacaatatgcgtcggtctgtctagagcccaaagttcctgctctgggctctctcagaaacttcaggagatgttagaacaagtctttccagttagagaagaggagtggtgataaagcccactgaccttcacacattct

SEQ ID NO: 8 - protein, 26 aa, Homo sapiens
ARVKMPEQEYEFPEPRNLNNNMRRSV

SEQ ID NO: 9 - DNA, 269 bp, Homo sapiens
atgcaagagtcaagatgccggagcaggaatatgaattccctgagccgcgaaatctcaacaacaatatgcgtcggccttcttccccccggaagtggtactctccaatcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattct

SEQ ID NO: 10 - protein, 57 aa, Homo sapiens
ARVKMPEQEYEFPEPRNLNNNMRRTQQGTSSLPPSSKAFCLEPKVPALGSLRNFRRC

SEQ ID NO: 11 - DNA, 335 bp, Homo sapiens
atgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttaggccttctgtctagagcccaaagttcctgctctgggctctctcagaaacttcaggagatgttagaacaagtctttccagttagagaagaggagtggtgataaagcccactgaccttcacacattct

SEQ ID NO: 12 - protein, 101 aa, Homo sapiens
ARVKMPEQEYEFPEPRNLNNNMRRPSSPRKWYSPIKGKLDALWVLLRKGYDRVSVMRPQPGDTGRCINLGLLSRAQSSCSGLSQKLQEMLLEQVFPVREEW

SEQ ID NO: 13 - DNA, 20 bp, Artificial
  FWD primer dor amplification of region 901-1040 of SEQ ID NO1
acagggtcctctgcagcttc

SEQ ID NO: 14 - DNA, 20 bp, Artificial
  REV primer for amplification of region 901-1040 of SEQ ID NO1
catgctgacctggagtgcag

SEQ ID NO: 15 - DNA, 21 bp, Artificial
  FWD primer for amplification of region 1387-1950 of SEQ ID NO1
atgcaagagtcaagatgcccg

SEQ ID NO: 16 - DNA, 20 bp, Artificial
  REV primer for amplification of region 1387-1950 of SEQ ID NO1
agaatgtgtgaaggtcagtg

SEQ ID NO: 17 - DNA, 21 bp, Artificial
  FWD primer for amplification of region 901-1145 of SEQ ID NO3
acagggtcctctgcagcttca

SEQ ID NO: 18 - DNA, 21 bp, Artificial
  REV primer for amplification of region 901-1145 of SEQ ID NO3
ctattccatgcaagcagctgt

SEQ ID NO: 19 - DNA, 22 bp, artificial
  FW primer for amplification all isoforms TEM8
acagggtcctctgcagcttcaa

SEQ ID NO: 20 - DNA, 24 bp, Artificial
  RV primer for amplification all isoforms TEM8
gtcagaacagtgtgtggtggtgat

SEQ ID NO: 21 - DNA, 24 bp, Artificial
  FW primer for amplification all isoforms CMG2
gtgtttattgtgttggtgtccttg

SEQ ID NO: 22 - DNA, 21 bp, Artificial
  RV primer for amplification all isoforms CMG2
gacaatctgaaattcctcccc

SEQ ID NO: 23 - DNA, 22 bp, Artificial
  FW primer for amplification TEM8 isoform 3
ggcatgaaagctgcactccagg

SEQ ID NO: 24 - DNA, 22 bp, Artificial
  RV primer for amplification TEM8 isoform 3
ccatgcaagcagctgttgtggg

(19) United States
(12) Patent Application Publication
(10) Pub. No.: US 2009/0304728 A1
(43) Pub. Date: Dec. 10, 2009

(54) TEM8 GENE, EXPRESSION FORMS AND DIAGNOSTIC AND THERAPEUTIC USES THEREOF

(75) Inventors: Antonio Concetti, Falerone (IT); Franco M. Venanzi, Camerino (IT)

Correspondence Address: LAHIVE & COCKFIELD, LLP;FLOOR 30, SUITE 3000, ONE POST OFFICE SQUARE, BOSTON, MA, 02109, US

(73) Assignee: BIOSOOFT S.R.L., Castelraimondo (IT)

(21) Appl. No.: 12/306,182

(22) PCT Filed: Jun. 26, 2007
(86) PCT No.: PCT/EP07/56357

§ 371 Date: May 8, 2009

(30) Foreign Application Priority Data
Jun. 27, 2006 (IT) RM 2006 A000337

(51) Int. Cl. A61K 39/00; C12Q 1/68; C07H 21/04; C12N 15/00; C12N 1/00; C12N 5/08; C07K 14/00; C07K 16/18; C07H 21/02; G01N 33/53; A61P 35/00

(52) U.S. Cl. 424/185.1; 435/6; 536/24.31; 536/24.33; 435/320.1; 435/243; 435/366; 530/350; 530/388.1; 530/389.7; 536/23.1; 435/7.1

(57) ABSTRACT

The present invention relates to the modulation of a gene known as “tumor endothelial marker 8 (TEM8)” and refers to the differential levels with which the gene is expressed, in its variants, in cells exhibiting angiogenic and migratory properties such as dendritic cells and metastatic tumor cells. The present invention further relates to the use of gene and polypeptide sequences of TEM8 as instruments of diagnosis and prognosis of pathological inflammatory angiogenesis and of the metastatic potential of tumor cells. The invention further relates to the use of the same gene and polypeptide sequences as direct therapeutic instruments, and to the use in immunogenic compositions or in vaccines apt to induce an immune response against cells overexpressing TEM8 gene products. Furthermore, the present invention relates to screening methods for identifying agonists and antagonists of TEM8 activity in any polynucleotide and/or polypeptide variant thereof to be used in prevention or therapy treatments. Lastly TEM8 expression profiles in in vitro expanded DCs from cancer patients intercepts responsiveness to cancer immunotherapy.

### DESCRIPTION

The present invention relates to the modulation of the expression of a gene known as “tumor endothelial marker 8 (TEM8)”, also known, in one of its splice variants, as receptor 1 of anthrax toxin, and refers to the differential levels with which the gene is expressed, in its variants, in cells exhibiting angiogenic and migratory properties such as dendritic cells and metastatic tumor cells. The present invention further relates to the use of gene and polypeptide sequences of TEM8 as instruments of diagnosis and prognosis of pathological inflammatory angiogenesis and of the metastatic potential of tumor cells. The invention further relates to the use of the same gene and polypeptide sequences as tool for the prediction of clinical response in immunotherapy of cancer patients. The invention further relates to the use of the same gene and polypeptide sequences as direct therapeutic instruments (e.g. iRNA, or peptides working as a decoy), and to the use in immunogenic compositions or in vaccines apt to induce an immune response against cells overexpressing TEM8 gene products. Lastly, the present invention relates to screening methods for identifying agonists and antagonists of the activity of TEM8 in any polynucleotide and/or polypeptide variant thereof to be used in prevention or therapy treatments.

### BACKGROUND OF THE INVENTION

Inflammation is a complex set of interactions among soluble factors and cells that can arise in any tissues in response to traumatic, infectious, post-ischemic or toxic injury. The process normally leads to recovery from infection and to healing. However if targeted destruction and assisted repair are not properly phased, inflammation can lead to persistent tissue damage by leukocytes and lymphocytes.

Recent data have expanded the concept that inflammation is a critical component of tumor progression. Indeed many cancer (about 15%) arise from sites of chronic inflammation and it is now clear that tumor-microenvironment, which is largely orchestrated by inflammatory cells, is indispensable to foster neoplastic growth and metastatic disease (Cossuens L M & Werb Z. 2002. Nature. 420: p 860-867. Insight Review Inflammation).

The growth of blood vessels (a process known as angiogenesis) is essential for organ growth and repair. An imbalance in this process contributes to numerous malignant, inflammatory, and ischemic disorders. Indeed, the importance of a deregulated angiogenesis for the tumor growth and metastatic spreading is universally recognized. Recently, the possibility that tumor-associated immunocells contribute to tumor vascularization has been reported (Adriana A, et al. Cancer Research 65, 10637-10641, Dec. 1, 2005).

Comparison of gene expression patterns of endothelial cells derived from normal and tumor blood vessels is an essential tool for understanding tumor angiogenesis. Although normal and tumor endothelium are highly related, tumor endothelium does exhibit qualitative and quantitative differences in gene expression patterns compared to endothelia derived from normal tissues. In one analysis, an array of over 46 gene transcripts were specifically elevated in tumor-associated endothelium (St. Croix B, et al. Science 2002, 289 (5482): 1197-1202). Among these genes are those encoding for tumor endothelials markers (TEMs). Indeed TEMs, while undetectable in normal quiescent vessels, are expressed both in tumor vasculature and in physiological angiogenesis (e.g. corpus luteum, granulation tissues of healing wounds, and embryogenesis. One of such genes, TEM8, is especially intriguing because it is not expressed in normal adult angiogenesis (see above). Three different version of TEM8 gene have been described (alternative splicing): Splicing variant 1 (TEM8.1), variant 2 (TEM8.2), variant 3 (TEM8.3. TEM8.1 was recently identified as the anthrax toxin receptor 1 (ATRX1) (Bradley K A, et al. Nature. 2001. 414 (6860): 225-229). Another member of the ATRX family, capillary morphogenesis protein 2 (CMG2/ATRX2) has been identified (Scobie H M et al. Proc. Natl Acd Sci USA. 2003. 100 (9):5170-5174).

Although TEM8 gene expression has been linked to tumor angiogenesis, large-scale expression monitoring and bioinformatics suggest (Novatchkova N & Eisenhaber F. Bioassy 2001. 23: 1159-1174) that TEM8 could be more generally expressed in different types of cells involved in extracellular matrix-remodelling and migration processes (e.g. leukocytes, endothelial cells, invasive cancer cells.

Cell migration is a specific property of invasive/metastatic cancer cells, of endothelial cells involved in angiogenetic processes, but also of innate immunocells like the dendritic cells (DCs), the most important professional cells of antigen processing and presentation to the immune system.

Ex vivo expanded DCs are currently applied as cellular vaccine (immune therapy) for cancer patients. Most commonly, DCs are generated by culturing blood derived monocytes (Mo-DCs) from patients in the presence of granulocytes-macrophage colony stimulating factor (GM-CSF) and IL-4, loaded with tumor antigens, and exposed to inflammatory signals (i.e. LPS, CD40L or Poly I:C.) to induce final maturation (Gilboa E. J. Clin. Invest. 117:1195-1203 (2007)). Most recent clinical trials of DC therapy for melanoma and renal cellular cancer (RCC) utilize DC matured in a cocktail of TNFα/IL-1β/IL-6 and Prostaglandin E2 (PGE2-mDCs). The rationale for including PGE2 in the maturation protocol is to endow the ex vivo-generated DCs with the capacity to migrate to lymphoid tissues, and to enhance T-cell activation potential (Luft T, et al. 2002 Blood. 100:1362-1372.) Nevertheless, when response rate in clinical trials were evaluated, PGE2-matured DC vaccines did not seem to be more effective compared with DC matured otherwise. Thus, whatever the maturation process, it appears extremely difficult to predict the efficacy of the antitumor therapy in vivo, which is characterized by high variability and low treatment response rates.

Tumor cells have co-opted some signaling molecules of the innate immunosystem to promote angiogenesis, migration and metastasis (Cossuens L M & Werb Z. 2002, Nature 420: p860-867: Insight Review Inflammation). However, in the current state of the art there are no molecular signatures that link these programs altogether. It is intended that tacking such specific gene-expression profiling to the clinic will outperform the conventional criteria utilized to evaluate the outcome of anti angiogenetic treatments in vivo, as well as to predict effectiveness of anti-cancer immunotherapy currently in use and/or under clinical development.

### SUMMARY OF THE INVENTION

The present invention is based on the discovery that the pro-angiogenic and migratory processes typical of metastatic tumor cells and dendritic cells are accompanied by expression or overexpression of the TEM8 gene in its variants. Therefore, the invention meets the above-indicated demands by singling out in the TEM8 gene a specific marker of pathologic inflammatory angiogenesis of the condition and the destiny of dendritic cells (DC) in connection to the pathologic angiogenesis and of the migratory and metastatic properties of cancer cells.

The invention is also based on the surprising discovery that in dendritic cell(DC)-based vaccination of cancer patients, the TEM8 expression profile evidences a significant correlation between high TEM8 mRNA levels and vaccination failure (i.e. progressive disease).

Accordingly, the present invention provides nucleic acids deriving from TEM8, comprising its alternative splicing products and products due to post-transcriptional modification, as well as relevant sequences of mRNA transcripts and amino acid sequences. Such proteins and/or mRNA are associated to: i) a pro-angiogenic activity at the inflammation site; ii) a pro-angiogenetic and migratory activity of the dendritic cells; iii) the migratory properties of tumor cells; iv) responsiveness to cellular DC vaccination in cancer advanced patients

Hence, main object of the invention is a method of diagnosis of tumor forms or states related to the onset of tumor forms, selected from pathologic inflammatory angiogenesis, tumor angiogenesis, high metastatic and/or migratory ability of tumor cells and of dendritic cells, comprising steps wherein it is detected, on a biological specimen, the activation and the extent of expression of the TEM8 gene or of regions thereof, in any one of its variants due to different splicing or post-transcriptional modification.

A second object of the invention is a method of prognosis of tumor, inflammatory and/or neoangiogenic states, as well as a method to monitor their therapeutic treatments, in which it is determined, on a biological specimen, the presence and the extent of expression of the TEM8 gene or of regions thereof, in any one of its variants.

A third object of the invention are genetic probes capable of hybridizing with specific regions of the TEM8 gene in all of its variants or with sequences exhibiting at least 95% homology therewith, and PCR primers for determining TEM8 gene variants linked to tumor forms.

A fourth object of the invention are TEM8 gene expression products, due to any different splice variant or post-transcriptional processing variant or their homologous sequences exhibiting at least 90% homology for use as diagnostic or therapeutic agents.

A fifth object is a method for selecting cancer patients, even in advanced phase, suitable to be responsive to cancer immunotherapy.

Further objects of the invention are immunogenic compositions comprising the TEM8 gene set in a plasmid vector suitable for genetic immunization, or one or more expression products of the TEM8 gene, capable of inducing an immune response against cells overexpressing the TEM8 gene, poly- or monoclonal antibodies specific for TEM8 gene expression products, specifically in the therapeutic treatment of inhibiting the pathologic inflammatory angiogenesis, the tumor neoangiogenesis, the metastatic and/or migratory ability of tumor cells and of dendritic cells, and as diagnostic reagents.

Other objects of the invention will be evident in the light of the detailed description hereinafter.

The advantages entailed in the invention are those of providing information of diagnostic, prognostic and therapeutic value, by means of the detecting of expression of TEM8 in the forms of its transcripts and/or its polypeptides from pathological specimens of inflammatory angiogenesis. A further advantage is that of detecting the presence of metastatic cells in tumor specimens from primary tissues, and the micrometastases from lymph nodes.

### BRIEF DESCRIPTION OF THE FIGURES

FIG. 1: the figure illustrates the results of ELISA assay for determination of VEGF in the isoforms 165 and 121 secreted by dendritic cells: immature, iDC; matured with cytokine cocktail (IL-6, IL-1β, TNF-α) in the presence of PGE2, mDC; matured in the absence of PGE2, mDC-PGE2; matured in the presence of Poly I:C in substitution of PGE2, mDC+PolyIC.

Detecting was performed by Pierce Biotechnology Kit, following the manufacturer's instructions.

FIG. 2: the figure illustrates the results of RT-PCR analysis of CMG2 transcripts (panel A) and of TEM8 (panel B) on immature dendritic cells (iDC) and matured with a cocktail containing PGE2 (mDC). Mw represents reference molecular weights.

Numbers denote the different patients from which dendritic cells originate. Letter C denotes the control in the absence of template.

On the bottom portion of each panel, PCR products of a reference gene GADPH are highlighted.

Arrows denote electrophoretic travel positions of the GMC2 fragments for panel A and of TEM8 for panel B.

FIG. 3: the figure illustrates the results of Real-Time RT-PCR for determination of the expression levels of TEM8 and CMG₂ in connection to the maturation from precursor monocytes (Mo) to mature DC cells (mDC) and in connection to the maturation cocktail (CTK) used (cytokines+PGE₂ or cytokines+Poly IC).

1) TEM8 transcripts of iDC vs. Mo; 2) TEM8 transcripts of mDC (cytokines+PGE₂) vs. immature dendritic cells (iDC); 3) CMG2 transcripts of mDC (cytokines+PGE₂) vs. iDC; 4) TEM8 transcripts of mDC (cytokines+PolyI:C) vs. iDC.

Calibrators; Mo(1) and iDC (2, 3 and 4). Bars denote mean values of 5 determinations, and lines denote minimum and maximum value of determinations for each group.

FIG. 4: the figure illustrates the results of Real-Time RT-PCR expression of TEM8 and of CMG2 in MDA-MB231 cells, as compared to that in ZR75-1 cells taken as calibrator and after normalization as compared to GADPH.

Dark-grey and light-grey unframed bars refer to TEM8 expression in MDA-MD231 and ZR75-1 cells kept for 48 h under growth conditions in a complete medium or in the absence of serum, respectively; framed bars refer to CMG2 expression for the same cells and under the same culture conditions.

FIG. 5: the figure illustrates the reactivity of anti-TEM8 antibodies produced via DNA vaccination with different plasmids integrating the nucleotide sequences SEQ ID NO: 4 SEQ ID NO: 5 and SEQ ID NO: 6. Productions of antibodies specific for TEM8 recombinant proteins were highlighted with Western blotting analysis against TEM8 recombinant protein.

FIG. 6: Quantitative PCR profiles of TEM8 and CMG2 gene expression (all mRNA isoforms) following exposure to maturation cocktail versus immature DCs=a cohort (n=21) of melanoma and renal cell cancer (RCC) patients treated with autologous DC vaccine. Dots, individual patients; Bars, mean fold increase, (mfi) TEM8 (mfi=7,6; SD=8) and CMG2 expression (mfi=2,9; SD=3) in matured DC vs immature DCs

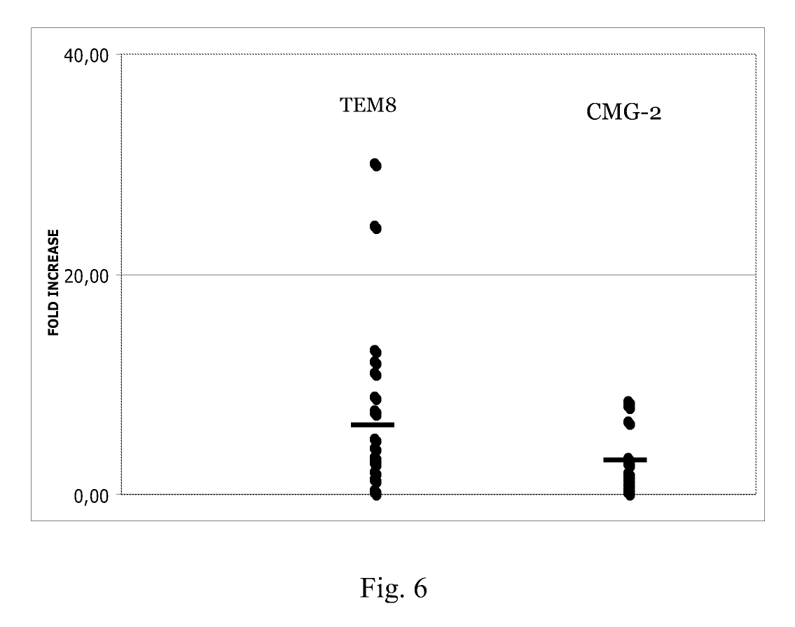

FIG. 7: Fold increase in TEM8 and CMG2 gene expression as measured by real Time PCR in patient population clustered for the responsiveness to DC treatment. NR, no response; R, response. Dots and bars as described in FIG. 7. NR TEM8 expression (mfi=13,2; SD=8,2); R TEM8 expression (mfi=1,9; SD=1,3). NR CMG2 expression (mfi=2,9; SD=3); R CMG2 expression (mfi=3,2; SD=3). **TEM8 values NR vs R, p=0.0018. CMG2 values NR vs R, not significant. Broken line, at about 5 fold increase, represents cutoff TEM8 gene expression increase discriminating R and NR patients.

FIG. 8: Q-RT PCR relative expression of TEM8.3 (TEM8 splicing isoform 3, white bars) vs TEM8 all isoforms (black bars) in PGE2 matured DCs from different non responsive cancer patients.

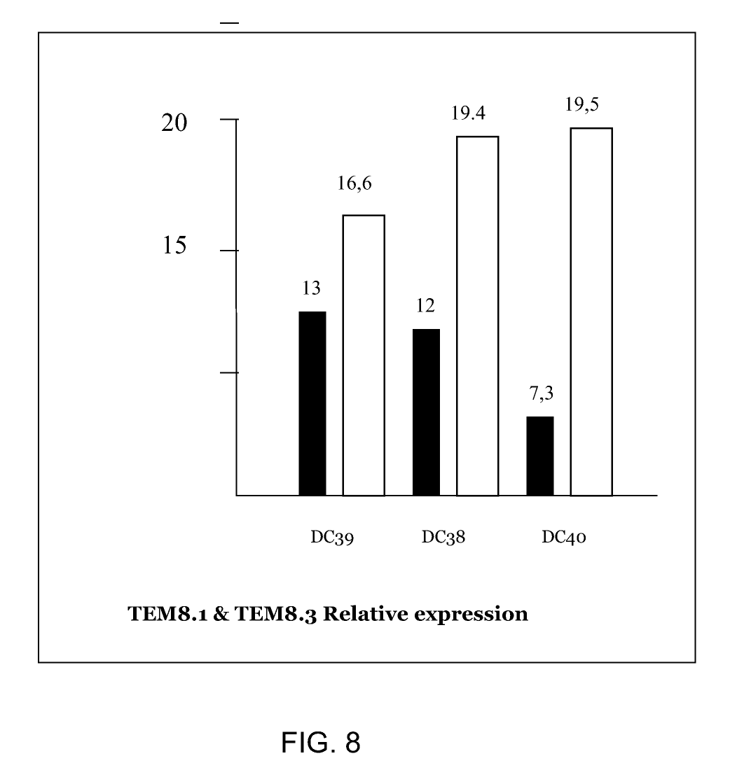

### DETAILED DESCRIPTION

Human “Tumor endothelial marker 8” (TEM8) gene is described in literature and three different variants thereof are known, due to alternative splicing modes.

Human TEM8 variants share the same amino-terminal extracellular portion, but differ in length and in the sequence of their cytosol regions.

Splice variant 1 (TEM8.1) is the longer and it is the original cDNA of TEM8 encoding a 564-aa protein, with a proline-rich long cytoplasmic tail. The nucleotide sequence between SV1 positions 144 and 1950 is reported hereinafter as SEQ ID NO: 1 (Gene Bank accession number AF_{—}279145), whereas the corresponding 564-aa sequence is reported as SEQ ID NO: 2. Splice variant 2 (TEM8.2) encodes a 368-aa protein with a short cytoplasmic tail. The nucleotide sequence between positions 144 and 1454 of the cDNA is reported hereinafter as SEQ ID NO: 3 (Gene Bank accession number NM_{—}053′34), whereas the corresponding sequence of 368 encoded amino acids is reported as SEQ ID NO: 4.

Splice variant 3 (TEM8.3) encodes a protein identical to the other two in most of the extracellular domain, and containing no membrane-anchoring sequence. The cDNA sequence of the SV3 variant between positions 144 and 2143 is reported hereinafter as SEQ ID NO: 5 (Gene Bank accession number NM_{—}018153), whereas the corresponding sequence of 317 encoded amino acids is reported as SEQ ID NO: 6.

Moreover, in accordance with the present invention in the TEM8 gene there have been highlighted high-variability zones comprising the SV1 gene portions delimited between positions 901-1040 and 1387-1950, and the SV3 variant portion delimited by positions 901 and 1145.

These zones were amplified by operating on different tumor cells via RT-PCT, using the primer pairs having sequence SEQ ID NO: 13 (FW) and SEQ ID NO:14 (RV) or SEQ ID NO:15(FW) and SEQ ID NO:16 or SEQ ID NO:17 (FW) and SEQ ID NO:18 (RV) or SEQ ID NO: 19 (FW) and SEQ ID NO: 20 (RV) or SEQ ID NO: 23(FW) and SEQ ID NO: 24 (RV).

Thus, there have been singled out new variant forms of the TEM8 gene, exhibiting, in zone 1387 to 1950 of SV1 and 901 to 1145 of SV3, relevant sequence deletions and/or mutations never observed before. Among these, new variants have been isolated, having, in the region starting from position 1387 of SEQ ID NO:1, three new sequences denoted as SEQ ID NO:7, SEQ ID NO:9 and SEQ ID NO:11. The amplification products thus obtained encode expression products having a polypeptide sequence comprising the sequences denoted as SEQ ID NO: 8, SEQ ID NO:10 and SEQ ID NO:12.

Without binding the invention to scientific theories not yet fully confirmed, it has to be stressed that the new variants of the TEM8 gene have been observed and isolated in tumor cells. Hence, these variants could likely be linked to tumor situations specific of a certain cell typology, or of a certain development phase of the tumor onset process. Therefore, the nucleic sequences of the present invention not only are generally useful as tumor markers, but may be useful in the accurate diagnosis of specific tumor forms or of tumor onset-linked precancerous forms, such as the pathologic inflammatory angiogenesis, the tumor angiogenesis, the metastatic and/or migratory ability of tumor cells and the migratory ability of dendritic cells.

Therefore, the method of diagnosis according to the invention is based on the detecting of the presence and/or expression or overexpression of the TEM8 gene in all its variants due to a different type of splicing or to a different post-transcriptional processing. Specific embodiments of the invention are diagnostic methods capable of recognizing in biological specimens the presence of cDNAs comprising the specific sequences denoted as SEQ ID NO:1; SEQ ID NO:3; SEQ ID NO:5.

However any alternative cDNA sequence differing from those seen above, yet obtained as amplification product of the extracellular domain (or portion thereof) or the intracellular domain of the TEM8 gene, in particular of the cDNA portions delimited by positions 901 and 1040 or 1387 and 1950 of sequence SEQ ID NO:1, or 901 and 1145 of sequence SEQ ID NO:5, are equally useful in a diagnostic method according to the invention. An example of such alternative sequences are sequences SEQ ID NO: 7, SEQ ID NO:9 and SEQ ID NO: 11 (FW) as well as any other amplification product obtained by RT-PCR using the primer pairs having sequences SEQ ID NO: 13 (FW) and SEQ ID NO:14 (RV) or SEQ ID NO:15(FW) and SEQ ID NO:16 or SEQ ID NO:17 (FW) and SEQ ID NO:18 (RV) or SEQ ID NO: 19 (FW) and SEQ ID NO: 20 (RV) or SEQ ID NO: 23(FW) and SEQ ID NO: 24 (RV).

Lastly, useful in the methods of the invention are all those sequences of nucleic acids having at least 95% homology and/or the ability to hybridize under high stringency conditions, with TEM8 and any variant or above-indicated fragment thereof, e.g. a single-strand DNA, an mRNA or an interfering RNAi.

Evaluation of the presence and the expression level of the TEM8 gene in its variants is performed through genetic probes or through agents capable of detecting the corresponding expression products.

Detection with Genetic Probes

Genetic probes are DNA or RNA sequences, usually single-strand, capable of hybridizing under certain stringency conditions with the TEM8 gene cDNA, in particular with the portions identifying the gene exons. Preferred probes are those capable of hybridizing under high stringency conditions, as defined in the examples, with the nucleotide sequences SEQ ID NO:1; SEQ ID NO:3; SEQ ID NO:5; SEQ ID NO:7; SEQ ID NO:9; SEQ ID NO:11, or with any TEM8 gene sequence PCR-amplified by using the primer pairs having sequences SEQ ID NO: 13 (FW) and SEQ ID NO:14 (RV) or SEQ ID NO:15(FW) and SEQ ID NO:16 or SEQ ID NO:17 (FW) and SEQ ID NO:18 (RV) or SEQ ID NO: 19 (FW) and SEQ ID NO: 20 (RV) or SEQ ID NO: 21(FW) and SEQ ID NO: 22 (RV) or SEQ ID NO: 23(FW) and SEQ ID NO: 24 (RV) or with the corresponding RNA transcripts. Such probes are usually labeled with molecules or reporter elements capable of highlighting the hybridation complex and introduced into the probe by known techniques such as PCR, recombination or enzymatic techniques. Suitable marker substances are nucleotides containing radioactive elements such as P³²-dNTP or S³⁵-dNDP or fluorescent or chemoluminescent substances.

Alternatively, probes may be highlighted after formation of the hybridation complex by suitable probe-specific antibodies.

Expression Products Detection

In a further embodiment of the invention, expression of the TEM8 gene in its variants is determined through detecting, on the biological specimen, the presence of the expression products of the cDNAs seen above. Such expression products are polypeptides having sequences selected from: SEQ ID NO:2; SEQ ID NO:4; SEQ ID NO:6; SEQ ID NO:8; SEQ ID NO:10; SEQ ID NO:12 or selected from all polypeptide sequences corresponding to PCR amplification products of the TEM8 gene using the primer pairs having sequences SEQ ID NO: 13 (FW) and SEQ ID NO:14 (RV) or SEQ ID NO:15(FW) and SEQ ID NO:16 or SEQ ID NO:17 (FW) and SEQ ID NO:18 (RV) or SEQ ID NO: 19 (FW) and SEQ ID NO: 20 (RV) or SEQ ID NO: 21(FW) and SEQ ID NO: 22 (RV) or SEQ ID NO: 23(FW) and SEQ ID NO: 24 (RV).

Of course, the methods seen hereto are useful not only for diagnostic purposes, but also for prognostic ones, and for purposes of assessing the effectiveness of therapeutic treatments aimed at the care of tumor and/or inflammatory states, and therefore to the prognosis of said states. In accordance with this aspect of the invention, it is determined on a patient's biological specimen, one or more times during the therapeutic treatment or at the end thereof, the presence and the extent of expression of the TEM8 gene or regions thereof, in any one of the variants due to different splicing or post-transcriptional modification, controlling over time the variations of the observed results.

Biological specimens on which the methods of diagnosis of the invention are conducted are human blood, synovial, pleural, bioptic collections, or collections of tumor tissues or samples of in vivo and ex vivo dendritic cells.

Proteins expressed from TEM8 gene variants, as well as their fragments and derivatives, are useful as immunogens for the production of poly- or monoclonal antibodies or functional antibody fragments.

Antibodies

The antibodies according to the invention are used both in diagnostic methods for the recognition, in biological specimens, of the expression products of the TEM8 gene in its variants, and in therapeutic treatment methods.

Antibodies useful in the methods of the invention directly bind TEM8 polypeptide sequences, eliminating or altering the functions thereof both by direct biochemical action and by effecting immunological action, via complement or via cytotoxic cells. Such functions are: the function in neoangiogenesis processes, the immunological function of dendritic cells or the migratory and metastatic function of tumor cells. Among the anti-TEM8 antibodies provided by the present invention, there are encompassed those that, by binding the TEM8, act as analytic or diagnostic instrument for in vitro and in vivo detection of TEM8.

Antibodies specific for the various expression products can be obtained by the conventional techniques well-known to a person skilled in the art, through animal immunization with the whole protein, protein portions or peptides, preferably bound to carrier proteins potentiating their immunogenic activity. Attainment of monoclonal antibodies through production of hybridoma lines is performed in accordance with methods detailed in the literature. Alternatively, laboratory animals can be immunized with DNA vaccines comprising a plasmid or a viral expression vector containing one of the TEM8 nucleotide sequences of the invention, optionally bound to a second sequence encoding a carrier protein.

Once introduced in the host cell, the plasmid or vector will express the protein or hybrid protein capable of stimulating antibody production, as described in the examples.

Primer

Further aspects of the invention are specific PCR or RT-PCR primers allowing to amplify domains of the splice variants SV1, SV2 and SV3 of the TEM8 gene, characterized by high variability. Examples of such primers are represented by sequences SEQ ID NO:13 (FW) and SEQ ID NO:14 (RV), amplifying SV1 sequence 901-1040, or by sequences SEQ ID NO:15 (FW) and SEQ ID NO:16 (RV), amplifying SV1 sequence 1387-1950, or by sequences SEQ ID NO 17 (FW) and SEQ ID NO: 18 (RV), amplifying SV3 sequence 901-1145. Additional primers are SEQ ID NO: 19 (FW) and SEQ ID NO: 20 (RV) or SEQ ID NO: 23(FW) and SEQ ID NO: 24 (RV) amplifying isoform 3.

Cloning or Expression Vectors

In accordance with methods exhaustively described in the literature, all nucleotide sequences of the invention may be introduced in suitable cloning and expression vectors for the production of corresponding recombinant products in host cells, as described in the examples. TEM8 nucleotide sequences are flanked by suitable control sequences directing and regulating their transcription and translation. Suitable host cells are prokaryotic or eukaryotic cells, in particular animal or human cells transformed by the vector containing the sequences of interest.

Therapeutic Treatments

Lastly, the nucleotide and polypeptide materials of the invention derived from the different variants of the TEM8 gene, as well as the corresponding antibodies, find application as medicaments in the treatment of tumor forms or of states related to the onset of tumor forms, such as: the pathologic inflammatory angiogenesis, the tumor angiogenesis, the metastatic and/or migratory ability of the tumor cells and the migratory ability of the dendritic cells.

As active nucleotide principles, there are used interfering RNAs, antisense RNA, or other nucleic material capable of inhibiting or modulating TEM8 gene expression, as well as polypeptides working as decoy.

Alternatively, there are used as active principles, along with pharmacologically acceptable excipients, polypeptide expression products in immunogenic compositions capable of inducing an immune response against cells overexpressing TEM8 gene products.

Accordingly, the present invention finds application in the characterization of human mammary tumor cells, in order to evaluate their metastatic potential and their ability to express the TEM8 gene, both in vivo and in ex vivo specimens.

Moreover, the invention finds application in the characterization of human dendritic cells, particularly yet not exclusively intended for cell vaccine production.

In fact ex vivo expanded dendritic cells (DCs) are currently applied as vaccine for cancer patients. Most commonly, DCs are generated by culturing blood derived monocytes (Mo-DCs) from patients in the presence of granulocytes-macrophage colony stimulating factor (GM-CSF) and IL-4, followed by exposure to inflammatory signals (i.e. LPS, CD40L or Poly I:C.) to induce final maturation. Most recent clinical trials of DC therapy for melanoma and renal cell cancer (RCC) utilize DC matured in a cocktail of TNFα/IL-1β/IL-6 and Prostaglandin E2 (PGE2-mDCs). Whatever the maturation process, it appears extremely difficult to predict the efficacy of the antitumor therapy in vivo, which is characterized by high variability and low treatment response rates in dendritic cell-based immunotherapy of advanced cancer.

Significantly, immature DCs (iDCs) from melanoma and RCC patients, matured with the standard cocktail of TNFα, IL-1β, IL-6 and prostaglandin E2, (PGE2-mDCs), while secreting comparable amounts of vascular endothelial growth factor A (VEGF-a), displayed widely different levels of tumor endothelial marker 8 (TEM8) gene-activity. (Range fold increase PGE2-mDCS vs iDCs from 0.1-30).

Surprisingly, the TEM8 expression data utilized as a stratification factor for tumour response, evidenced a significant correlation (p<0.001) between high TEM8 expression (>5 fold increase over basal immature DCs mRNA level) and vaccination failure (i.e. progressive disease)

In particular, the appearance of TEM8 phenotype associates with a high increase (>15 fold) in the level of TEM8 transcription, while it has a negligible effects on CMG2 expression in the all population of patients.

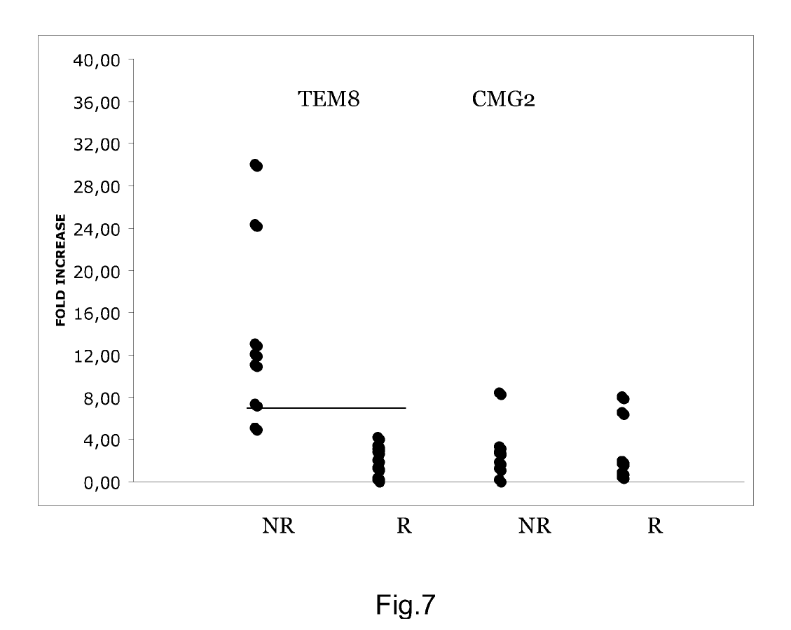

TEM8 increased expression in PEG2 matured dendritic cells (mDCs) versus immature DCs, spread from 1 to 30 folds. Surprisingly it has been observed that a cut off of about 5 folds as regard to the enhancement in the TEM8 expression level in mature DCs, is capable of discriminate between patients having more than 5 folds, who are clinically unresponsive (progressive disease) to DC treatment (p<0.0018), and patient with less than 5 folds, who are responsive (i.e. complete, partial and mixed responses, stable disease) (FIG. 7).

Accordingly, the invention provides the instruments needed for the detecting of the differential expression of polynucleotide and polypeptide sequences of the TEM8 gene and splicing products thereof in human dendritic cells, in connection to the responsiveness or unresponsiveness of the patient to cancer immunotherapy.

Moreover, the invention finds application in the characterization of the propagation state of chronic inflammation process and in the evolutive potential of the same. In this scope, it finds application also as indicator of the therapeutic effectiveness of antinflammatory drugs currently in use and under development in therapy.

In a further application thereof, the present invention provides the formulation and the methods for use of TEM8 polypeptides and/or polynucleotides in immunogenic compositions for inducing immunity against target cells such as the tumor cells, the endothelial cells and any other cell overexpressing TEM8 products.

There are also provided diagnostic methods for detecting diseases associated to TEM8 gene overexpression in all of its variants, or of the related polypeptides, comprising the use of such a detecting as prognostic marking method in tumors, and in any other disease characterized by chronic inflammation; methods for treating said pathologies are provided as well.

Lastly, the invention allows the development of screening methods for identifying novel TEM8 ligand molecules, mainly yet not exclusively for pharmacological use.

The invention is illustrated hereinafter with all experimental details in the following examples.

### Example 1

### Dendritic Cell Pro-Angiogenetic Phenotype Assignment

The pro-angiogenetic potential of dendritic cells matured, under clinical grade conditions, with the most common cocktails used in clinical trials, i.e. IL1β, IL6, TNFα and PGE2, or with the same cocktail having replaced PGE2 with Poly I:C, was evaluated by ELISA analysis, by measuring the levels of VEGF secreted into the culture medium.

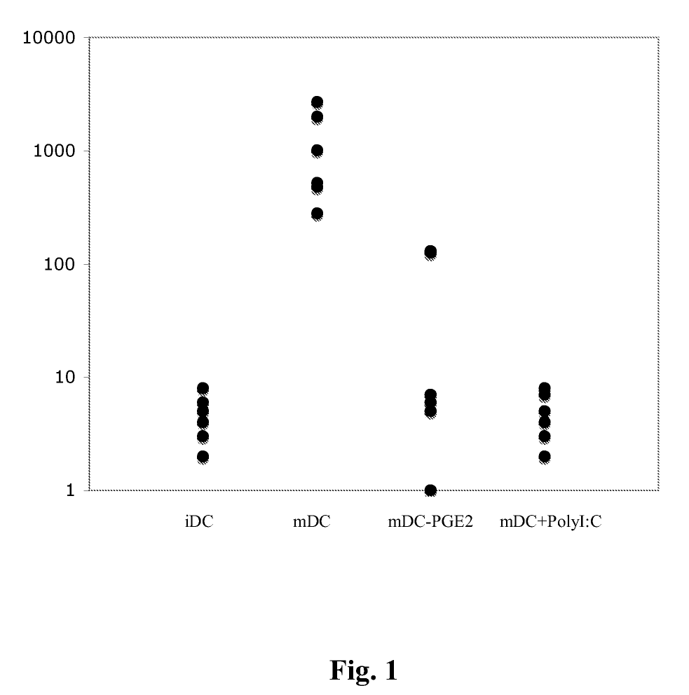

VEGF is produced by alternative splicing of a single gene into multiple isoforms, among which the most common ones are VEGF 121 and VEGF 165. FIG. 1 illustrates VEGF production (forms 165 and 121 are both recognized by antibodies provided with the Endogen human VEGF ELISA Kit, Pierce Biotechnology Inc.) in cultured DC supernatants. Analysis was performed as follows: supernatants of immature DC, of DC matured with the full cocktail of cytokines and of tumor cells untreated and treated with the full cocktail, were harvested and used for VEGF quantitation, according to the protocol provided by the kit.

For this purpose, the anti-human VEGF 165 antibody was adhered to the 96-well plate, where it captured the VEGF present in the specimens added to the plate. Addition of 50 μl specimen into the wells was followed by 2 hours of incubation at room temperature. After 3 rinsings with Wash Buffer, 100 μl biotynilated antibody were added to each well; such a 1-hour incubation was followed by 3 rinsings and 30 min incubation with Streptavidin-HRP Reagent. A subsequent addition of 100 μl substrate allowed to measure absorbance at 450 nm.

FIG. 1 shows VEGF production by dendritic cells matured in full (cytokines+PGE2) cocktail. Negligible VEGF production was observed in immature dendritic cells or in dendritic cells matured with Poly I:C.

### Example 2

### IL-12 Anti-Angiogenetic Factor Production Inhibition in DC Matured with Poly I:C

Since VEGF has been reported to inhibit IL-12 production and immune response Th1 differentiation, the ability of DC, matured with the PGE2-containing cocktail, to produce biologically active IL-12 was evaluated.

Interleukin 12 p70 (IL-12p70), a heterodimer comprised of subunits p35 and p40, is the major cytokine in the induction of a TH-1 response and of a powerful anti-angiogenetic activity. Conversely, in the absence of p35 expression, the DC-secreted monomer and homodimer of IL-12p40 act as IL-12 antagonists.

IL-12p40 and IL-12p35 determination was performed by means of real-time RT-PCR, according to the following procedures: at +48 hours, supernatants were harvested and stored at −20° C. until cytokine-measuring assays were performed. After discarding the culture medium, RNA extraction was performed.

Cells were lysed by incubation with a lysis buffer immediately inactivating the RNAse and creating appropriate binding conditions favoring RNA absorption to the silica membrane. Contaminant DNA was removed by a DNase I solution directly applied onto the silica membrane during the preparation. Simple rinsing steps with two different buffers remove salts, metabolites and macromolecular cell components. Pure RNA was eluted under low ionic strength conditions with RNase-free water.

RNA concentration was determined spectrophotometrically, by measuring absorbance at 260 nm, and RNA integrity was confirmed by electrophoresis on a 1.2% agarose gel.

1 μg total RNA was subsequently used to synthesize a single strand complementary DNA (cDNA) by RevertAid H Minus First Strand cDNA Synthesis Kit (Fermentas, Life Sciences).

The RNA (1 μg) was incubated with H₂O and 1 μl Oligo dT Primer (0.5 μg/μl) for 5 min at 70° C. 2 μl 10× Reaction Buffer, 2 μl RNAse inhibitor and 2 μl (10 nM) dNTPs mix were added to the reaction. The reaction mix was heated at 37° C. for 5 min.

Then, 1 μl RevertAid H Minus M-MuLV Reverse Transcriptase (200 u/μl) (final volume 20 μl) was added to the reaction, and incubated for 60 min at 42° C. The reaction was heated to 70° C. for 10 min to inactivate Reverse Transcriptase.

The resulting cDNA was used to determine TEM8 and CMG2 by Real-time.

Real-time RT-PCR was performed by using MX3000P Real-time PCR system (Stratagene) and BRILLIANT SYB Green QPCR Master mix according to the manufacturer's protocol. After initial denaturation for 10 min at 95° C., 40 cycles were performed with passages of 94° C. for 48 sec, 60° C. for 48 sec, and 72° C. for 48 sec, with fluorescence reading at the end of each cycle.

The following oligonucleotides were used as PCR primers: for TEM8, FW: ACAgggTCCTCTgCAgCTTCAA and Rev: gTCAgAACAgTgTgTggTggTgAT; for CMG2, FW: gTgTTTATTgTgTTggTgTCCTTg and Rev: gACAATCTgAAATTCCTCCCC. The primers amplify a 200-bp portion in the extracellular domain of TEM8 and of CMG2. Analyses were carried out with MxPro QPCR Software version 3.00 for MX3000P. The values obtained were within the linear range of a standard curve and were normalized to produce the same amount of messenger RNA (mRNA) of glyceraldehyde phosphate dehydrogenase (GAPDH) (Fw GADPH: CAACAgCgACACCCACTCCT and Rev GADPH: AggCCATgTgggCCATgA). All PCR products were analyzed by melting curve determination, as well as by agarose gel electrophoresis. The results reported in (Table A) indicate that DC matured in a cocktail with PGE2 yield high levels of IL-12p40 mRNA, yet not of p35, thereby inhibiting IL12-p70 production and shifting the balance between pro- and anti-angiogenetic factors in favour of angiogenesis. The same indication comes from P40 protein measuring by Elisa assay.

TABLE A
ELISA Analysis of immature and full
cocktail-matured DC supernatants.

| TEST ^{x} | | iDC | mDC |
|---|---|---|---|
| ELISA | IL-12 p40 * | 44 | >1350 |
| | IL-12 p70 ** | nd | nd |
| | IL-10 *** | 6.7 | 70 |
| | INFy *** | 5.16 | 14 |

^{x} pg × 10⁶ cells/ml;
*, **, *** referring to three different kits

Supernatants of immature DC and of DC matured with the cytokin cocktail were harvested and used for the quantitation of IL-12 p40 by ELISA assay (Biosource, Nivelles, Belgium) according to the protocol provided by the kit.

100 μl of the supernatants were added into the wells of the precoated plate and incubated for 2 hours at room temperature and under stirring. After 3 rinsings with Working Wash Buffer there followed a 2-hour incubation at room temperature and under stirring with the HRP-conjugated anti-IL-12p40 antibody. After 3 rinsings with Working Wash Buffer, 200 μl chromogenic solution were added; the plate was incubated for 30 minutes at room temperature, in the dark. Then, 50 μl Stop Solution were added and the reading at 450 nm was performed. Assay results are reported in Table A.

### Example 3

### TEM8 Gene Expression in Connection to Monocyte/Mature DC Differentiation

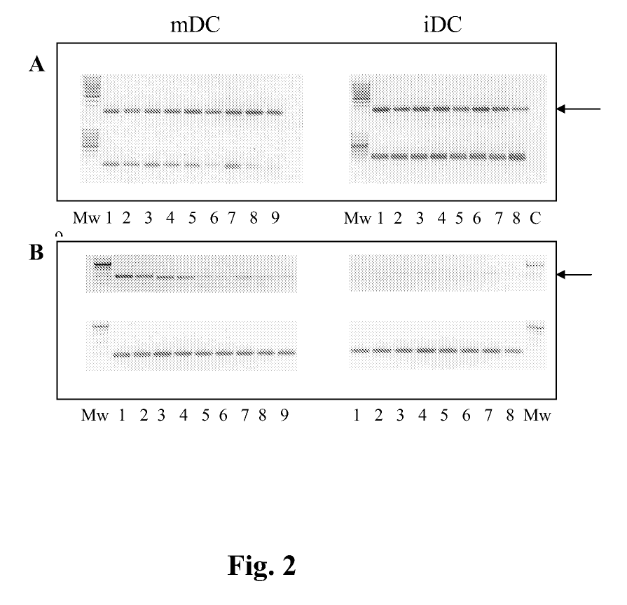

Conventional RT-PCR analyses show (FIG. 2) that, while the CMG2 gene is abundantly and evenly expressed in monocytes (Mo), in immature DC and in DC matured with PGE2-containing coktail, TEM8 expression is restricted to DC matured with PGE2.

The relative quantitation of gene expression operated by quantitative Real-Time PCR (Q-RT-PCR), confirms that both immature DC and precursor MO accumulate greater amounts of CMG2 transcripts as compared to those of TEM8 transcripts (Table B, Line 1 and 2), whereas there are no significant variations in the transcriptional activities of the two genes in going from the precursor Mo to immature DC (Table B, Line 3 and 4).

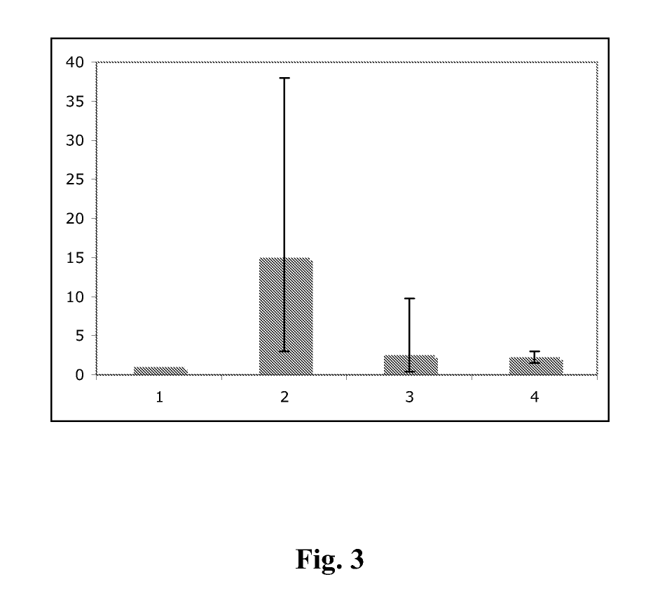

On the contrary, DC treated with PGE2-containing cocktail selectively increase TEM8 expression, more than 15-fold, as compared to the immature cells or the precursor Mo or the DC matured with Poly I:C (FIG. 3 and Table B, Line 5), thereby indicating that the increase in TEM8 transcription is strictly PGE2-dependent.

TABLE B
Real-time RT-PCR data on relative expression
of transcripts of CMG2 vs TEM8 in maturing
from monocytes to mature dendritic cells.

| CELLS | mRNA ratio | medium value (range) |
|---|---|---|
| Mos | CMG2 vs TEM8 | 143 (70-250) |
| iDCs | CMG2 vs TEM8 | 230 (50-461) |
| mDCs | CMG2 vs TEM8 | 150 (60-278) |

### Example 4

### Analysis of Expression of TEM8 and of CMG2 in Tumor Cells

Human metastatic mammary carcinoma cell lines ZR75-1 and MDA-MB231 were kept in culture under moist environment of 5% CO2, in Dulbecco's modified Eagles medium (D-MEM) (Cambrex) supplemented with 10% fetal bovine serum (FBS) (Cambrex), 1% L-glutamine, 1% penicillin/streptomycin.

The procedures for TEM8 and CMG2 expression determination in real-time RT-PCR are the same reported in Example 2.

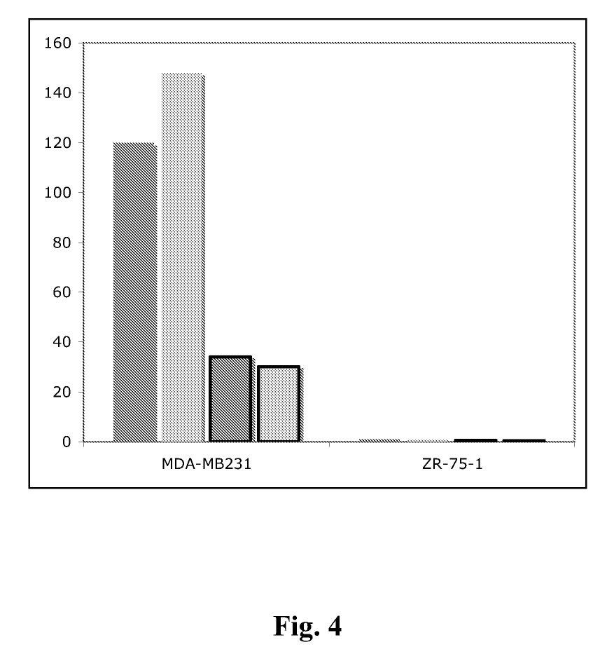

By using ZR75-1 as calibrator, it is detected a TEM8 expression level at least 150-fold higher in MDA-MB231 either in the absence or in the presence of serum. CMG2 expression in MDA-MB231 increases only 30-fold (FIG. 4).

### Example 5

### TEM8 Variants in Mammary Tumor Cells

By using the primers of ID seq 9 and 10, the corresponding region on Seq ID NO: 1 was PCR-amplified; following cloning in pGEM T easy vector and subsequent clone sequencing, 3 transcriptional variants were found, bearing variations of the deletion and frameshift type, whose sequences are denoted by SEQ ID n 4, 5 and 6, respectively.

### Example 6

### Construction of Eucaryotic Expression Vector for TEM8

The expression vector pcDNA3.1 (Invitrogen) was selected in order to clone the extracellular portion and the transmembrane domain of the TEM8 gene fused with the extracellular domain of the Flt-3 gene for the TEM8 sequence portion (Gene Bank accession number: 010229).

The portion of the TEM8 gene was obtained by PCR, using as template DNA a cDNA obtained from a total RNA extracted from tumors of FVB/233 mice transgenic for rat neu oncogene (Charles River).

For amplification, there were used the primers:

| | |
|---|---|
| FWm8F13 | gggggTACCggCCgCCgCgAggATgggggA |
| RVEcoRIm8 | ggTggAATTCCTAgCACAgCAAATAAgTgTCTTC |

with the following PCR conditions:

| | | | |
|---|---|---|---|
| 95° C. | 5′ | | |
| 95° C. | 1′ | | |
| 64° C. | 1′ | | |
| | | {close oversize brace} | 35 cycles |
| 72° C. | 2′ | | |
| 72° C. | 10′ | | |

The gene Flt-3 portion was obtained by PCR, using as template the plasmide pNGVL-mFL (Michigan, University) exaclty as described in Hung et al 2001.

Subsequently, the two PCR products and the vector were double-digested with the restriction enzymes (Fermentas) HindIII/KpnI (for the Flt-3), KpnI/EcoRI (for the TEM8), HindIII/EcoRI (for the vector) and, after purification performed with NucleoSpin Extract Kit (Macherey-Nagel), were cloned by using a T41igase (Fermentas). Then, the ligation product was transformed by electroporation (1 pulse at 2.5 kV for 2.5 msec) (Micropulser Electroporation Apparatus, Bio-Rad) of the prokaryiotic cells DH5α (Takara).

The plasmid, after a check by sequencing, was produced in large-scale using the Qiagen Plasmid Giga kit (Qiagen).

The plasmid thus obtained was used for animal immunization and anti-TEM8 antibodies production.

### Example 7

### Mice Immunization and Sera Collection

Balb/c mice were kept under pathogen-free conditions and in accordance to Ministry of Health Guidelines, at the stabularium of the INRCA Research Department of Ancona.

The immunization schedule consisted in three administrations into the femoral muscle, of 100 μg in 100 μl plasmid DNA described in Example 6, in physiological solution, 15 days apart from each other.

Blood was collected from the retroocular plexus in all mice before (preimmune serum) and at +15 days from the last recall (immune serum).

### Example 8

### TEM8 Recombinant Protein Production and Purification

Part of the gene for the TEM8 (13-375 Gene Bank accession number NM_{—}010229) was inserted in plasmid pGEM-T Easy Vector (Promega) by ligation; then, the ligation product was used to transform electrocompetent cells (DH5α strain of E. coli, TAKARA) with Biorad Micropulser.

Cells were seeded on Luria Broth Agar (LB) medium containing the antibiotic ampicillin. From colonies formed, the DNA was extracted according to Miniprep Qiagen Kit methods and then sequenced.

TEM8 DNA amplification and DNA transfer were performed by Gateway Cloning Technology. The cell-extracted DNA (plasmid pGEM-TEM8) was linearized with restriction enzyme SalI. The linearized pGEM-TEM8 was amplified by PCR using specific primers: AttB1bis and AttB2bis, essential in order to obtain a PCR product with attB1 and attB2 sites at the ends.

PCR mTEM8 program:

| | | | |
|---|---|---|---|
| 95° C. | 5′ | | |
| 95° C. | 1′ | | |
| 64° C. | 1′ | | |
| | | {close oversize brace} | 35 cycles |
| 72° C. | 2′ | | |
| 72° C. | 10′ | | |

Primers:

attB1bis-
ggggACAAgTTTgTACAAAAAAgCAggCTTgATgggCCgCCgCgAggATg
ggggA
attB2bis-
ggggACCACTTTgTACAAgAAAgCTgggTCgCACAgCAAATAAgTgTCTT
C

After product purification by gel elution, the two BP and LR reactions (Gateway) were performed in accordance to the provider's (GIBCO) instructions. Therefore, a transformation was effected by electroporation (Micropulser, BioRad) of electrocompetent TAKARA DH5α cells with the product of the BP Reaction; the Entry Clone proved correct by sequencing.

The primers used were:

| | |
|---|---|
| ATTL1- | TCgCgTTAACgCTAgCATggATCTC |
| ATTL2- | gTAACATCAgAgATTTTgAgACAC |

The conditions for sequencing were:

| | | | |
|---|---|---|---|
| 95° C. | 5′ | | |
| 96° C. | 10″ | | |
| | | {close oversize brace} | 30 cycles |
| 50° C. | 5″ | | |
| 60° C. | 4′ | | |

The Expression Vector yielded by Gateway was used to transfect the BL21 Star strain of E. coli. Transfected cells were grown in an O.N. culture at 37° C. and induced by 0.5M IPTG.

After induction, cultures were lysed; the obtained sample was loaded on HiTrap Chelating HP column (Pharmacia) in 20 mM sodium phosphate buffer+0.5M NaCl at pH7.8.

Elution was performed by stepwise decreasing of one pH unit, to pH4.0. The recombinant protein was eluted to pH≈6.0.

A further purification step was performed on FPLC, with Mono Q column in 20 mM sodium phosphate, pH8.00 with a continuous NaCl gradient from 0 to 0.3M. The recombinant protein eluted at the concentration of about 0.1M NaCl, with a symmetrical peak denoting the high degree of homogeneity, confirmed in 12.5% SDS-PAGE in which the protein, overloaded in the gel, showed a single electrophoretic band.

### Example 9

### Anti-TEM8 Antibodies Specificity

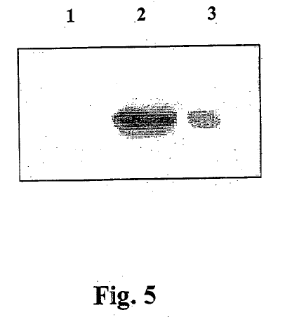

To evaluate the specificity of the antibodies obtained following anti-TEM8 genetic immunization, TEM8 recombinant protein was run on a 12.5 polyacrylamide and subsequently transferred on a nitrocellulose membrane. After incubation with the sera diluted 1:30 for 1 h at room temperature, the membrane was rinsed with PBST 3× for 5 min, then incubated with a peroxidase-conjugated anti-mouse antibody (Calbiochem) at a 1:3000 dilution for 1 h at room temperature. The reaction was highlighted on Kodak photographic plate, chemiluminescence-exposed and developed in the dark for 1-3 min with the Enhanced Chemiluminescence Kit, Amersham Life Science. The results reported in FIG. 5 show specificity to TEM8 of the antibodies produced.

### Example 10

### Fold Increase in TM8 and CMG2 Gene Expression in Patient Population Clustered for Responsiveness/Unresponsiveness to DC Cellular Vaccination

We retrospectively analyzed cryopreserved DC from 20 melanoma patients (stage IV) treated (phase I/II) with the same cells in vitro expanded, charged of autologous tumor antigens and matured with TNFα, IL-1β, IL-6 and PGE2 cocktail (Ridolfi R, Petrini M, Fiammenghi L, Stefanelli M, Ridolfi L, Ballardini M, Migliori G, Riccobon A. Improved overall survival in dendritic cell vaccination-induced immunoreactive subgroup of advanced melanoma patients. J Transl Med. Aug. 16, 2006;4:36.)

RNA Isolation and Complementary DNA Synthesis

Total RNA was isolated from monocytes, immature and mature human MoDCs, obtained from patients. The cells (5×10⁵) were lysed by incubation with a lysis buffer that immediately inactivates RNases and creates appropriate binding conditions which favour adsorption of RNA to the silica membrane. Contaminating DNA is removed by a DNase I solution which is directly applied onto the silica membrane during the preparation. Simple washing steps with two different buffers remove salts, metabolites and macromolecular cellular components. Pure RNA is eluted under low ionic strength conditions with RNase-free water. The concentration of RNA was determined spectrophotometrically by measuring absorbance at 260 nm and RNA integrity was confirmed by electrophoresis on a 1.2% agarose gel.

We used 1 μg total RNA for synthesis of first-strand complementary DNA (cDNA) by RevertAid H Minus First Strand cDNA Syntesis Kit (Fermentas, Life Sciences). In the complementary cDNA synthesis we used also the total human colon RNA (Ambion). The RNA (1 μg) was incubated with H₂O and 1 μl of Oligo dT Primer (0.5 μg/μl) for 5 minutes at 70° C. At the reaction were added 2 μl of 10× Reaction Buffer, the RNAsi inhibitor and 2 μl of 10 nM dNTPs mix. The reaction mixture was heated to 37° C. for 5 minutes. At the reaction was then added 1 μl of the RevertAid H Minus M-MuLV Reverse Transcriptase (200 u/μl) (final volume 20 μl) and incubated for 60 minutes at 42° C. Reaction was heated to 70° C. for 10 minutes to inactivate Reverse Transcriptase. The resulting cDNA was used for qualitative reverse-transcription polymerase chain reaction (RT-PCR) and for quantitative Real-Time PCR.

Quantitative Real-time RT-PCR was performed by means of the MX3000P Real-time PCR system (Stratagene) and the BRILLIANT SYB Green QPCR Master mix according to the protocol provided by the manufacturer. After initial denaturation for 10 minutes at 95° C., thermal cycling was performed for 40 cycles with steps of 94° C. for 48 seconds, 60° C. (62° C. for p40 and p35, 64° C. for p19) for 48 seconds, and 72° C. for 48 seconds, with the fluorescence being read at the end of each cycle. The following oligonucleotides were used as primers for the PCR:

| TEM8 (all isoforms) | |
|---|---|
| FW: | ACAgggTCCTCTgCAgCTTCAA |
| Rev: | gTCAgAACAgTgTgTggTggTgAT |
| CMG2 (all isoforms) | |
| FW: | gTgTTTATTgTgTTggTgTCCTTg |
| Rev: | gACAATCTgAAATTCCTCCCC |

In order to quantify the relative amounts of TEM8 mRNA isoform 3 over all isoforms Q-RT-PCR was performed using specific primers (ref: Christopher Premanandan, Michael D. Lairmore, Soledad Fernandez and Andrew J. PhippsQuantitative measurement of anthrax toxin receptor messenger RNA in primary mononuclear phagocytes Microbial Pathogenesis Volume 41, Issues 4-5, October-November 2006, Pages 193-198)

| TEM8.3 (Isoform 3) | |
|---|---|
| Fw: | GGCATGAAAGCTGCACTCCAGG |
| Rv: | CCATGCAAGCAGCTGTTGTGGG |

Q-RT-PCR reaction conditions: 50° C. for 40 min and 95° C. for 15 min for one cycle followed by 94° C. for 15 s, 49° C. for 20 s, 72° C. for 10 s and a 5 s acquisition at 79° C. for 50 cycles. The cycling conditions for the CMG2 RT-PCR were as follows: 50° C. for 40 min and 95° C. for 15 min for one cycle followed by 94° C. for 15 s, 49° C. for 20 s, 72° C. for 10 s and a 5 s acquisition at 75° C. for 50 cycles.

Analysis was performed with MxPro QPCR Software version 3.00 for MX3000P. The obtained values were within the linear range of a standard curve and were normalized to yield the same amount of glyceraldehyde phosphate dehydrogenase (GAPDH) messenger RNA (mRNA) (Fw GADPH: CAACAgCgACACCCACTCCT and Rev GADPH: AggCCATgTgggCCATgA). All PCR products were analyzed by determination of melting profiles as well as by agarose gel electrophoresis.

DNA Sequencing

To confirm the specific amplification of the extra cellular and transmembrane domain of human TEM8 and the portion of the extra cellular domain of human capillary morphogenesis protein 2, DNA sequencing was performed on PCR products from each different sample monocytes, iDC, mDC. The PCR bands were excised from the agarose gel, purified with Macherey-Nagel gel extraction columns and sequenced in both orientations. The sequencing reactions were carried out by MWG Biotech/M-Medical (Germany).

Results

Data reported in FIG. 1 show that the anti-inflammatory stimuli given by PGE2 is essential to the generation of a pro-angiogenic phenotype during the in vitro process of dendritic cell maturation performed either with the standard cytokine cocktail or with LPS. On the contrary, DCs maturation in absence of PGE2 or in presence of pro-inflammatory stimuli such as Poly I:C, strongly downregulated VEGF production. Pro angiogenic DCs phenotype may or may not associate with TEM8 gene-expression activity. Indeed only DCs matured with the standard cytokine cocktail showed a high increase (>13 fold) in the level of TEM8 transcription, while LPS+PGE2 matured DCs had negligible effects on TEM8 expression. Differently, CMG2 transcription profiles were basically unaffected by the type of maturation stimuli.

TEM8 increased expression in PEG2 matured dendritic cells versus immature DCs, spread from 1 to 30 folds. When a cut off of 5 regard to the enhancement in the TEM8 expression level was used, it was surprisingly find (FIG. 7) that all the patients with more than 5 folds were clinically unresponsive (progressive disease) to DC treatment (p<0.005), while all the others with less than 5 folds, were responsive (i.e. complete, partial and mixed responses, stable disease).

1-26. (canceled)

27. A method of diagnosis of tumor forms or states related to the onset of tumor forms, selected from pathologic inflammatory angiogenesis, tumor angiogenesis, metastatic and/or migratory ability of tumor cells and of dendritic cells, comprising steps wherein it is detected, on a dendritic cell specimen, the activation and the extent of expression of the TEM8 gene or of regions thereof, in any one of its variants due to different splicing or post-transcriptional modification.

28. The method according to claim 27, wherein TEM8 gene expression is detected through determination, on the biological specimen, of the presence of the cDNA sequences selected from: SEQ ID NO:1; SEQ ID NO:3; SEQ ID NO:5; SEQ ID NO:7; SEQ ID NO:9; SEQ ID NO:11, or of any TEM8 gene sequence PCR-amplified by using primer pairs having sequences: SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) of the presence of the corresponding RNA transcripts, or of the corresponding polypeptide expression products.

29. The method according to claim 28, wherein TEM8 gene expression is determined through detecting, on the biological specimen, the presence of expression products having peptide sequences selected from: SEQ ID NO:2; SEQ ID NO:4; SEQ ID NO:6; SEQ ID NO:8; SEQ ID NO:10; SEQ ID NO:12, or selected from the peptide sequences corresponding to PCR amplification products of the TEM8 gene, by using primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV).

30. The method according to claim 28, wherein TEM8 gene expression is detected through one or more genetic probes capable of hybridizing under high stringency conditions with a nucleotide sequence selected from SEQ ID NO:1; SEQ ID NO:3; SEQ ID NO:5; SEQ ID NO:7; SEQ ID NO:9; SEQ ID NO:11, or with TEM8 gene sequences PCR-amplified by using primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) or with sequences exhibiting at least 95% homology therewith.

31. A method of prognosis of tumor states or inflammatory states, wherein during or following the anti-tumor or anti-inflammatory treatment it is determined, on a dendritic cell specimen, the presence and the extent of expression of the TEM8 gene or of regions thereof, in any one of its variants, due to different splicing or to post-transcriptional modification.

32. A method of diagnosis of tumor forms or states related to the onset of tumor forms selected from pathologic inflammatory angiogenesis, metastatic and/or migratory ability of tumor cells and of dendritic cells, comprising steps wherein it is detected, on a biological specimen, the activation and the extent of expression of the TEM8 gene or of regions thereof, in any one of its variants due to different splicing or post-transcriptional modification.

33. The method according to claim 32, wherein TEM8 gene expression is detected through determination, on the biological specimen, of the presence of the cDNA sequences selected from: SEQ ID NO:1; SEQ ID NO:3; SEQ ID NO:5; SEQ ID NO:7; SEQ ID NO:9; SEQ ID NO:11, or of any TEM8 gene sequence PCR-amplified by using primer pairs having sequences: SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) of the presence of the corresponding RNA transcripts, or of the corresponding polypeptide expression products.

34. The method according to claim 33, wherein TEM8 gene expression is determined through detecting, on the biological specimen, the presence of expression products having peptide sequences selected from: SEQ ID NO:2; SEQ ID NO:4; SEQ ID NO:6; SEQ ID NO:8; SEQ ID NO:10; SEQ ID NO:12, or selected from the peptide sequences corresponding to PCR amplification products of the TEM8 gene, by using primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV).

35. The method according to claim 33, wherein TEM8 gene expression is detected through one or more genetic probes capable of hybridizing under high stringency conditions with a nucleotide sequence selected from SEQ ID NO:1; SEQ ID NO:3; SEQ ID NO:5; SEQ ID NO:7; SEQ ID NO:9; SEQ ID NO:11, or with TEM8 gene sequences PCR-amplified by using primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) or with sequences exhibiting at least 95% homology therewith.

36. The method according to claim 32, wherein the biological specimen is a blood, synovial, pleuric, bioptic sampling, or a sampling of tumor tissue or a dendritic cell specimen.

37. A method of prognosis of inflammatory states, wherein during or following the anti-inflammatory treatment it is determined, on biological specimen, the presence and the extent of expression of the TEM8 gene or of regions thereof, in any one of its variants, due to different splicing or to post-transcriptional modification.

38. A genetic probe for determining the presence and the extent of expression of the TEM8 gene capable of hybridizing under high stringency conditions with TEM8 gene regions having nucleotide sequence selected from: SEQ ID NO:1; SEQ ID NO:3; SEQ ID NO:5; SEQ ID NO:7; SEQ ID NO:9; SEQ ID NO:11, or with TEM8 gene sequences PCR-amplified by using primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) or with sequences exhibiting at least 95% homology therewith.

39. PCR Primers for determining TEM8 gene variants linked to tumour forms, comprising the following sequences: SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV).

40. A cloning or expression vector containing a nucleotide sequence selected from: SEQ ID NO:1; SEQ ID NO:3; SEQ ID NO:5; SEQ ID NO:7; SEQ ID NO:9; SEQ ID NO:11, or any one of the TEM8 gene sequences PCR-amplified by using the primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ D NO: 23(FW) and SEQ ID NO: 24 (RV), flanked by suitable sequences regulating the transcription and the translation thereof.

41. A prokaryotic or eukaryotic host cell modified by means of the vector according to claim 40.

42. An expression polypeptide of the TEM8 gene, obtained in a host cell according to claim 41.

43. The expression polypeptide of the TEM8 gene, comprising polypeptide sequences selected from SEQ ID NO:2; SEQ ID NO:4; SEQ ID NO:6; SEQ ID NO:8; SEQ ID NO:10; SEQ ID NO:12 or polypeptides corresponding to the TEM8 gene sequences PCR-amplified by using the primer pairs having sequences SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) or homolog sequences thereof having at least 90% homology.

44. An expression product according to claim 43 for use as diagnostic or therapeutic agent.

45. The product according to claim 44 in the diagnosis or therapeutic treatment of tumor forms or of states related to the onset of tumor forms, selected from pathologic inflammatory angiogenesis, tumor angiogenesis, metastatic and/or migratory ability of tumor cells and of dendritic cells.

46. An immunogenic composition comprising one or more expression products according to claim 43, capable of inducing an immune response against cells overexpressing TEM8 gene products, and a pharmaceutically acceptable excipient.

47. The immunogen composition comprising a vector according to claim 40, capable of inducing an immune response against cells overexpressing TEM8 gene products, and a pharmaceutically acceptable excipient.

48. A polyclonal or monoclonal antibody specific for the expression products according to claim 43.

49. The antibody according to claim 48 for use in the therapeutic treatment of inhibiting tumor states or states related to the onset of tumor forms selected from: the pathologic inflammatory angiogenesis, the tumor neoangiogenesis, the metastatic and/or migratory ability of tumor cells and of dendritic cells.

50. The antibody according to claim 48 for use as diagnostic reagent for the diagnosis of the pathologic inflammatory angiogenesis, the tumor neoangiogenesis, the metastatic and/or migratory ability of tumor cells and of dendritic cells.

51. A method of screening agonists or antagonists of TEM8 gene activity, comprising the step of treating cells expressing the TEM8 gene with the candidate agonist or antagonist and evaluating the levels of the RNA transcripts or of the expression products corresponding to sequences SEQ ID NO:1; SEQ ID NO:3; SEQ ID NO:5; SEQ ID NO:7; SEQ ID NO:9; SEQ ID NO:11.

52. A cDNA sequence selected from: SEQ ID NO:1; SEQ ID NO:3; SEQ ID NO:5; SEQ ID NO:7; SEQ ID NO:9; SEQ ID NO:11, or any TEM8 gene sequence PCR-amplified by using the primer pairs having sequences: SEQ ID NO:13(FW) and SEQ ID NO:14 (RV) or SEQ ID NO:15 (FW) and SEQ ID NO:16 (RV) or SEQ ID NO: 17 (FW) and SEQ ID NO:18 (RV), or SEQ ID NO: 19 (FW) and SEQ ID NO: 20 (RV) or SEQ ID NO: 23(FW) and SEQ ID NO: 24 (RV) or corresponding mRNA or iRNA transcripts, taken individually or in a mixture for use in the therapeutic treatment of tumor forms or of states related to the onset of tumor forms selected from pathologic inflammatory angiogenesis, tumor angiogenesis tumorale, metastatic and/or migratory ability of tumor cells and of dendritic cells.

53. A cDNA sequence selected from SEQ ID NO:7, SEQ ID NO:9, SEQ ID NO:11 or TEM8 gene sequence comprising one of these sequences, RNA transcripts and expression products thereof.

54. A method for selecting patients with advanced cancer eligible for dendritic cells vaccination comprising the step of in vitro quantifying TEM8 expression in PEG2-matured dendritic cells (PEG2-mDCs) from the patient and selecting those patients showing a relative TEM8 expression enhancement (matured DCs vs immature DCs) less than 5 folds, said patients being responsive to dendritic vaccines.

55. A method of evaluating the efficacy of an anti-angiogenic therapy in cancer patients comprising the step of in vitro quantifying the level of TEM8 expression in PGE2-mature dendritic cells (PEG2-mDCs) from the patients, comparing the observed expression level to the level of a previous determination, wherein decrease of TM8 expression indicates efficacy of the therapy.

56. The method according to claim 50, wherein the TEM8 gene is the TEM8 isoform 3.

57. The method according to claim 51, wherein the TEM8 gene is the TEM8 isoform 3.
